(19) United States
(12) Patent Application Publication
(10) Pub. No.: US 2019/0056373 A1
(43) Pub. Date: Feb. 21, 2019

(54) METHOD FOR PREDICTIVE DETERMINATION OF THE BEHAVIOR OF A REACTIVE MIXTURE INTENDED FOR OBTAINING A GEOPOLYMER, AND METHOD FOR OPTIMIZATION OF SAID GEOPOLYMER

(71) Applicants: UNIVERSITE DE LIMOGES, Limoges (FR); CNRS, Paris Cedex 16 (FR)

(72) Inventors: Ameni Gharzouni, LIMOGES (FR); Laëticia Vidal, Remoulins (FR); Sylvie Rossignol, RILHAC RANCON (FR); Elodie Prud'Homme, LYON (FR); Alexandre Autef, SAINT VICTURNIEN (FR); Emmanuel JOUSSEIN, LIMOGES (FR)

(73) Assignees: UNIVERSITE DE LIMOGES, Limoges (FR); CNRS, Paris Cedex 16 (FR)

(21) Appl. No.: 15/770,966

(22) PCT Filed: Oct. 27, 2016
(86) PCT No.: PCT/EP2016/075954

§ 371 Date: Apr. 25, 2018

(30) Foreign Application Priority Data
Oct. 27, 2015 (FR) 1560222

(51) Int. Cl. G01N 33/24; G01N 13/00; C04B 28/00; C04B 40/00

(52) U.S. Cl. CPC G01N 33/24; C04B 40/0032; C04B 28/006; G01N 13/00

(57) ABSTRACT

A method for determination of the behavior of a reactive mixture intended for obtaining a geopolymer. The reactive mixture comprises at least one aluminosilicate material. The method includes determining a proportion of amorphous phase of the at least one aluminosilicate material and determining a degree of wettability of the at least one aluminosilicate material. If the proportion of amorphous phase is greater than 45% and if the degree of wettability is situated in a range between 300 μg/l and 1400 μg/l, then the reactive mixture, formed by the reaction of the at least one aluminosilicate material with an alkaline solution, forms a geopolymer.

### RELATED APPLICATIONS

This application is a national phase of International Application No. PCT/EP2016/075954 filed on Oct. 27, 2016, which claims priority to French Application No. 1 560 222 filed on Oct. 27, 2015, the entirety of both of which are incorporated herein by reference.

### FIELD AND BACKGROUND OF THE INVENTION

The method according to the present invention relates to a method for determination of the behavior of a reactive mixture intended for obtaining a geopolymer.

The invention also concerns a method for optimization of said geopolymer.

In the following description geopolymers are understood to be binders obtained from mineral raw materials of different kinds and an alkaline solution or alkaline source. A polycondensation reaction makes it possible to polymerize the aluminosilicates which are obtained.

Unlike geopolymers, the raw materials for the production of cements essentially include limestone which supplies the lime and clays which supply in particular aluminum, silicon and iron oxide for producing clinker, the basis of the hydraulic binder which is cement. These materials generally have low contents of lime, production of which in a rotary kiln involves very high energy consumption since the necessary temperatures are of the order of 1500° C.

Geopolymers are materials which have the advantage that they require little energy in order to ensure their synthesis. The industry is searching for products which have low carbon emissions during production and which can replace materials which have a highly negative ecological impact, such as cement.

Moreover, geopolymers can be partially recycled, without treatment, by simple addition to processes for producing other geopolymers.

The difficulty in this field is to determine the properties of the material which will be obtained from a composition based on selected mineral raw materials. The possibilities are, of course, infinite, but the industry is searching for a method which makes it possible to determine the properties of the product obtained, or even to enable optimization of certain characteristics of said product, as required.

There are two materials which influence the finished product:
an aluminosilicate source having a chemical composition and provided with physico-chemical parameters,
an alkaline solution or alkaline source which supplies the alkaline cations and the aqueous characteristics.

### BRIEF DESCRIPTION OF THE INVENTION

The method according to the invention proposes to determine the intrinsic characteristics of the aluminosilicate source, making it possible to produce a geopolymer. In particular, the invention relates to a method for determination of the behavior of a reactive mixture intended for obtaining a geopolymer, said reactive mixture comprising at least one aluminosilicate material, the method comprising the following steps:
determining the proportion of amorphous phase of the at least one aluminosilicate material,
determining the degree of wettability of the at least one aluminosilicate material,

and, if the proportion of amorphous phase is situated in a range between 25 and 99% and if the degree of wettability is situated in a range between 300 and 1400 μg/l, then the reactive mixture formed by the reaction of the at least one aluminosilicate material with an alkaline solution is a geopolymer.

### BRIEF DESCRIPTION OF THE DRAWINGS

The method according to the invention is now described in detail, with reference to the different drawings which illustrate the steps of the method, according to a particular non-limiting embodiment. In the drawings:

FIG. 1 shows a graph indicating the proportion of amorphous phase for 17 samples of raw material,

FIG. 2 shows a graph representing the wettability of the 17 samples,

FIG. 3 shows a graph representing variations of the Si/Al ratio,

FIG. 4 shows a curve representing the reactivity of the solution during variations of Si/M, M being an alkali, more particularly Na or K,

FIG. 5 shows a diagram indicating the values for strain at break for each sample which has formed a geopolymer, 7 days after the consolidated geopolymer is obtained,

FIGS. 6A to 6C show three representations of obtaining a geopolymer from an alkaline solution SK1 based on potassium which is not very reactive, and a very reactive aluminosilicate composition MP1, a very reactive aluminosilicate composition MP2 and an aluminosilicate composition MP3 which is not very reactive,

FIGS. 7A to 7C show three representations of obtaining a geopolymer from an alkaline solution SK2 based on potassium which is very reactive, and a very reactive aluminosilicate composition MP1, a very reactive aluminosilicate composition MP2 and an aluminosilicate composition MP3 which is not very reactive,

FIGS. 8A to 8C show three representations of obtaining a geopolymer from an alkaline solution SNa based on sodium which is very reactive and highly viscous, and a very reactive aluminosilicate composition MP1, a very reactive aluminosilicate composition MP2 and an aluminosilicate composition MP3 which is not very reactive.

### DETAILED DESCRIPTION OF THE INVENTION

In the case of a raw material intended for obtaining a geopolymer within the meaning of the present invention, the method according to the invention consists of determining a first factor intrinsic to the raw materials, a factor capable of leading to a geopolymer when it is in a given range, namely the proportion of amorphous phase.

The proportion of amorphous phase is the proportion of material in which the initial lattice of the material has been destructured.

The base materials are chosen from among aluminosilicates including kaolins or also metakaolins which are kaolins which are dehydroxylated, generally by heating, and actually have a highly disorganized lattice.

In order to determine the range correlated with the first factor of the method, tests are conducted on the basis of numerous samples, in this case samples of raw materials comprising:
a colloidal silica solution with a varying proportion of silica from 12 to 18%, expressed by weight in relation to the total weight,
an alkaline solution with a ratio of 6% to 53% of potassium oxide, expressed by weight in relation to the total weight, and a water content of 35 to 80%, expressed by weight in relation to the total weight.

Thus 17 samples are studied on this basis with a view to obtaining compositions capable of producing geopolymers.

Once the composition is obtained, it is placed in a mold, preferably of a closed type, in order to give it the profile required for the tests. This mold is preferably of a closed type because this avoids dehydration occurring too rapidly at the interface, as this dehydration can be a source of cracking of the samples, which is prejudicial to the tests to be carried out.

The results obtained are assembled in the graph of FIG. 1.

In reality it will be noted that three different classes of material are obtained:
the raw materials MP1-MP10 lead to the formation of geopolymer materials,
the raw materials MP11, MP13-MP15 lead to the formation of gels with an anisotropic contraction of 3% after consolidation,
the raw materials MP12, MP16 and MP17 do not exhibit any consolidation and remain in the form of a stratified material.

Thus, all the compositions do not lead to geopolymers.

The proportion of amorphous phase is predetermined by x-ray diffractometry (XRD) and a correlation is noted between the proportion of amorphous phase and the results obtained.

The first 10 materials which have led to a geopolymer material being obtained have a proportion of amorphous phase greater than 45% and more particularly greater than 80%, as FIG. 1 shows. Nevertheless, this criterion does not appear sufficient since certain raw materials have a high proportion of amorphous phase and do not form a geopolymer, such as MP12.

This proportion of amorphous phase corresponds to the proportion of material which is capable of reacting in the presence of an alkaline solution, and which makes it possible to obtain aluminates and silicates, once the dissolution is carried out in the presence of an alkaline solution.

The method according to the present invention provides for analysis of a second factor which has a marked influence on the abilities of an aluminosilicate material to form a geopolymer, namely the wettability.

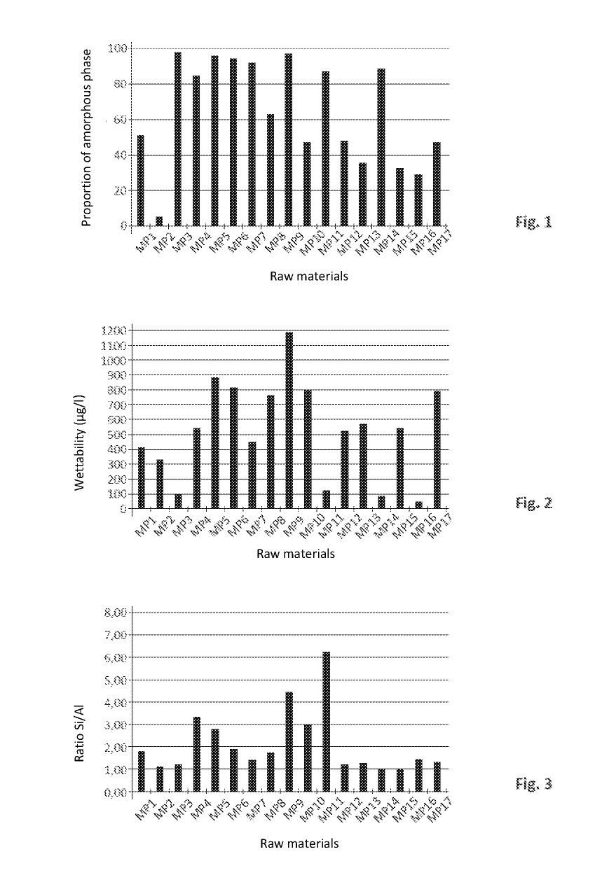

To this end, a wettability test is carried out on the samples and these tests give the results on the graph of FIG. 2.

The wettability test consists of using a layer of raw material having a given thickness and the wettability corresponds to the quantity of water which generates a drop at the surface without it being absorbed. It will be noted that the test makes it possible to differentiate once again between the raw material compositions as envisaged for the formation of a geopolymer.

Some of the materials MP1-MP10 which have led to the formation of a geopolymer exhibit a wettability between 300 and 1400 μg/l, more particularly between 500 and 1400 μg/l.

Also, the method according to the present invention consists of analyzing the first and second factors, and if the first factor is greater than 45% and if the second factor is between 300 and 1400 μg/l, then the initially proposed material composition will lead to the formation of a geopolymer. Preferably, if the first factor is greater than 80% and if the second factor is between 500 and 1400 μg/l, then the geopolymer obtained exhibits improved mechanical parameters.

According to an improvement in the method according to the present invention, a third factor can make it possible to refine the prediction of formation of a geopolymer, i.e. the analysis of the Si/Al ratio.

An analysis by any appropriate means, such as an apparatus for analysis by x-ray fluorescence, makes it possible to measure out the two compounds Si and Al and to determine the ratio.

The results are shown on the graph of FIG. 3.

It has been found that the materials initially tested, MP1-MP10, which lead to a geopolymer also have a value of the Si/Al ratio between 0.5 and 4.4, preferably between 0.9 and 1.2.

Thus, a diagram can be established with the two first factors and a ternary zone by addition of the third factor, again defining, in a more predictive manner, the range for formation of a geopolymer.

According to the present invention, the predictive method provides that, in the case of an aluminosilicate composition situated in the ternary zone, it is possible to obtain a geopolymer.

Nevertheless, the alkaline solution which is used in combination with the aluminosilicate composition situated in the ternary zone in order to obtain a geopolymer is a basic solution comprising alkaline ions in an aqueous medium, which is generally the least expensive and the most easily available. Mention may be made of in particular of soda and potash.

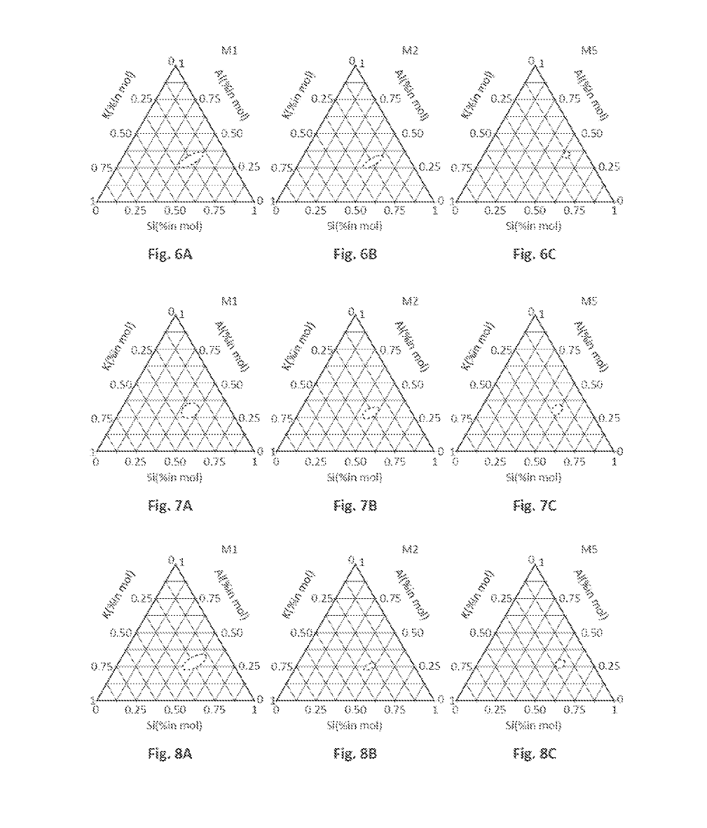

The concentration and the viscosity of the alkaline solution, for example, can influence the formation of a geopolymer as shown by the examples of FIGS. 6A to 6C, 7A to 7C and 8A to 8C.

It will be noted on the three representations of FIGS. 6A to 6C that the formation of a geopolymer is associated with the reactivity of the available aluminosilicate phase because the zones of formation of geopolymers are similar and extensive for the two most reactive compositions M1 and M2 and this zone of formation of geopolymers is limited for the less reactive composition M5. In fact, in these reactions the alkaline solution based on potassium SK1 has a low reactivity.

On FIGS. 7A to 7C, the zones of formation of geopolymers are substantially identical for the three aluminosilicate compositions, including the composition which is not very reactive, which shows that the high reactivity of the alkaline composition compensates for the poor reactivity of said composition.

On FIGS. 8A to 8C, in the case of the highly reactive alkaline composition, it will be noted that the aluminosilicate composition MP2 is not very conducive to the formation of geopolymers whilst this composition MP2 is highly reactive.

In this case, the aluminosilicate composition MP2 has a high wettability value, which limits the diffusion of the sodium ions in the composition and therefore the formation of a geopolymer.

The composition MP5 itself has a low reactivity, but it will be noted that the zone of formation of geopolymers is nevertheless extensive because of the high reactivity of the alkaline solution and because the wettability value thereof is not too high.

The reactivity of the aluminosilicate compositions and of the alkaline solutions is a supplementary factor for the prediction of formation of geopolymers.

Thus, the pH value should be greater than 12.5 for a relationship 0.5<Si/Al<4.4.

The geopolymers obtained can be optimized, as a function of the previously established results, according to the parameters to be improved.

In fact, the geopolymers have numerous applications, and these applications can necessitate reinforcement of certain parameters.

Mention may be made in particular of the essential parameters, which are resistance to compression, resistance to water and resistance to fire.

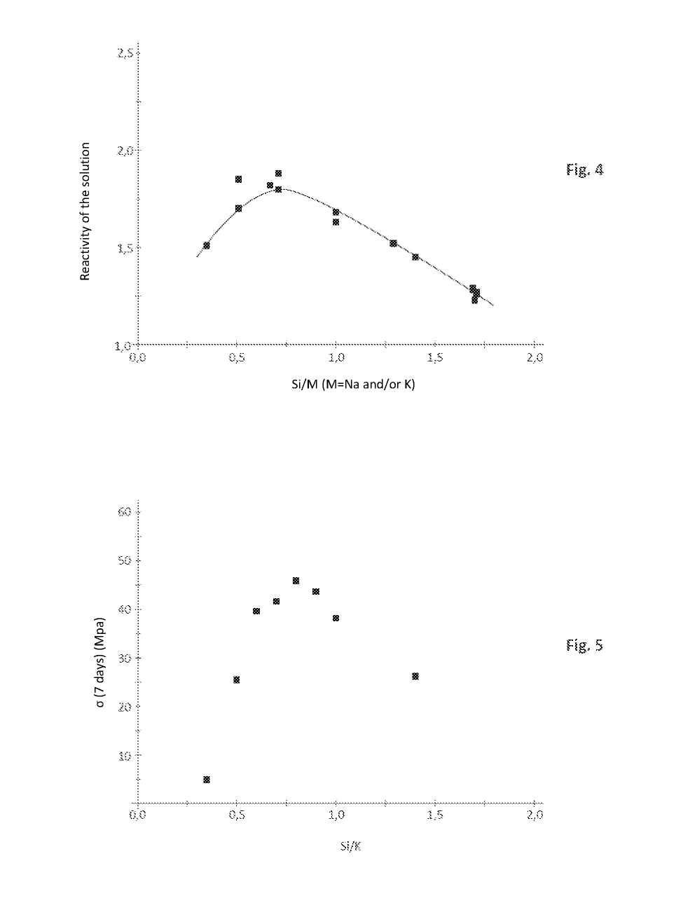

To this end, a fourth factor can be taken into account, namely the ratio Si/M of which the curve is indicated on FIG. 4, M being an alkali and more particularly Na or K.

Thus, this ratio Si/M must be between 1.7 and 0.35 in order to obtain a suitable geopolymer.

More particularly, this ratio is between 1.0 and 0.7.

The higher the proportion of amorphous phase is, the higher the mechanical resistance is, as is indicated on the graph of FIG. 5.

It should also be noted that certain geopolymers obtained exhibit a mechanical resistance which varies over time and exhibits an increase in the mechanical resistance.

A fifth factor can be taken into account, the proportion of solvent, namely the percentage of water in the alkaline solution.

The relationship is such that the higher the ratio Si/M is, the more the proportion of water is decreased.

Thus, for a ratio Si/M=1.7 a water ratio of 30% will be used, and for a ratio Si/M=0.35 a water ratio of 80% will be used.

As a function of the desired properties, it may be stated that:
the higher the values of the proportion of amorphous phase are, the higher the mechanical properties will be, and
the higher the proportion of Si/M is, the higher the mechanical properties will be, regardless of the alkaline cation chosen.

If the object is to search for a geopolymer which has a good resistance to water, then it is necessary to prioritize a high proportion of Si/M.

Such a geopolymer can also be used as insulating material and, in this case, agents are added to the composition without this modifying the predicted properties or the optimized properties.

As an example of a possible agent, mention may be made of a blowing agent to reduce the density of the material, for example a blowing agent selected from among the silicas containing metallic silicon or silicon carbide.

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

1. A method for determination of a behavior of a reactive mixture intended for obtaining a geopolymer, said reactive mixture comprising at least one aluminosilicate material, wherein the method comprises the following steps:
determining a proportion of amorphous phase of the at least one aluminosilicate material,
determining a degree of wettability of the at least one aluminosilicate material, and,
if the proportion of amorphous phase is greater than 45% and if the degree of wettability is situated in a range between 300 μ/l and 1400 μg/l, the reactive mixture, by a reaction of the at least one aluminosilicate material with an alkaline solution, forms is a geopolymer.

2. The method for determination of the behavior of a reactive mixture intended for obtaining a geopolymer, according to claim 1, wherein the amorphous phase is greater than 80%.

3. The method for determination of the behavior of a reactive mixture intended for obtaining a geopolymer, according to claim 2, wherein the degree of wettability is between 500 μg/l and 1400 μg/l.

4. The method for determination of the behavior of a reactive mixture intended for obtaining a geopolymer according to claim 1, further comprising:
determining a ratio of Si/Al of the at least one aluminosilicate material and,
if said ratio of Si/Al is between 0.5 and 4.4, then the reactive mixture, by the reaction of the at least one aluminosilicate material with the alkaline solution, forms is a geopolymer.

5. The method for determination of the behavior of a reactive mixture intended for obtaining a geopolymer according to claim 1, further comprising:
determining a ratio of Si/Al of the at least one aluminosilicate material and,
if said ratio of Si/Al is between 0.9 and 1.2, then the reactive mixture, by the reaction of the at least one aluminosilicate material with the alkaline solution, forms a geopolymer.

6. The method for determination of the behavior of a reactive mixture intended for obtaining a geopolymer according to claim 5, wherein when the ratio of Si/Al is between 0.5 and 4.4 and when the alkaline solution has a pH higher than 12.5, then the reactive mixture, by the reaction of the at least one aluminosilicate material with said alkaline solution, forms a geopolymer.

7. The method for determination of the behavior of a reactive mixture intended for obtaining a geopolymer according to claim 1, wherein, in the case of a geopolymer, in order to improve one or more mechanical properties of said geopolymer, a ratio of Si/M, wherein M represents an alkaline cation, is situated between 0.35 and 1.7.

8. The method for determination of the behavior of a reactive mixture intended for obtaining a geopolymer according to claim 1, wherein, in the case of a geopolymer, in order to improve one or more mechanical properties of said geopolymer, a ratio of Si, wherein M represents an alkaline cation, is situated between 1.0 and 0.7.

9. The method for determination of the behavior of a reactive mixture intended for obtaining a geopolymer, according to claim 1, wherein the degree of wettability is between 500 μg/l and 1400 μg/l.

10. The method for determination of the behavior of a reactive mixture intended for obtaining a geopolymer according to claim 4, wherein when the ratio of Si/Al is between 0.5 and 4.4 and when the alkaline solution has a pH higher than 12.5, then the reactive mixture formed by the reaction of the at least one aluminosilicate material with said alkaline solution forms a geopolymer.

11. The method for determination of the behavior of a reactive mixture intended for obtaining a geopolymer according to claim 10, wherein, in the case of a geopolymer, in order to improve one or more mechanical properties of said geopolymer, a ratio of Si/M, wherein M represents an alkaline cation, is situated between 0.35 and 1.7.

12. The method for determination of the behavior of a reactive mixture intended for obtaining a geopolymer according to claim 10, wherein, in the case of a geopolymer, in order to improve one or more mechanical properties of said geopolymer, a ratio of Si/M, wherein M represents an alkaline cation, is situated between 1.0 and 0.7.

13. The method for determination of the behavior of a reactive mixture intended for obtaining a geopolymer according to claim 7, wherein, in the case of a geopolymer, in order to improve one or more mechanical properties of said geopolymer, a ratio of Si/M, wherein M represents an alkaline cation, is situated between 0.35 and 1.7.

14. The method for determination of the behavior of a reactive mixture intended for obtaining a geopolymer according to claim 7, wherein, in the case of a geopolymer, in order to improve one or more mechanical properties of said geopolymer, a ratio of Si/M, wherein M represents an alkaline cation, is situated between 1.0 and 0.7.
